(19) United States
(12) Patent Application Publication
(10) Pub. No.: US 2026/0294863 A1
(43) Pub. Date: Oct. 1, 2026

(54) METHOD OF TREATING FIBROTIC-RELATED CONDITION

(71) Applicant: YISSUM RESEARCH DEVELOPMENT COMPANY OF THE HEBREW UNIVERSITY OF JERUSALEM LTD., Jerusalem (IL)

(72) Inventor: Marta WEINSTOCK-ROSIN, Jerusalem (IL)

(21) Appl. No.: 19/490,069

(22) PCT Filed: Jun. 5, 2024
(86) PCT No.: PCT/IL2024/050554

§ 371 Date: Dec. 4, 2025

Related U.S. Application Data
(60) Provisional application No. 63/471,008, filed on Jun. 5, 2023.

(51) Int. Cl. A61K 31/27; A61K 31/4045; A61P 11/00; A61P 17/00

(52) U.S. Cl. CPC A61K 31/27; A61K 31/4045; A61P 11/00; A61P 17/00

(57) ABSTRACT

Provided herein is a pharmaceutical composition comprising a therapeutically effective amount of an antioxidant for use in a method for treating or preventing a fibrotic-related condition in a subject in need thereof.

### CROSS-REFERENCE TO RELATED APPLICATIONS

This application claims the benefit of priority of U.S. Provisional Patent Application No. 63/471,008, titled “METHOD OF TREATING FIBROTIC-RELATED CONDITION” filed 5 Jun. 2023, the contents of which are incorporated herein by reference in their entirety.

### FIELD OF INVENTION

The present invention is in the field of fibrotic-related condition.

### BACKGROUND OF THE INVENTION

Systemic sclerosis (SSc) is a complex autoimmune disease with heterogeneous clinical manifestations and varying clinical courses that affects many organs.

In SSc, the response to injury that normally leads to tissue regeneration is subverted into fibrosis as a result of the accumulation of excessive extracellular matrix (ECM) causing disruption of tissue architecture and loss of functional integrity. Such excessive deposition of connective tissue components occurs in organs in response to injury caused by ischemia, oxidative stress and autoimmune reactions. Fibrotic tissue remodeling is usually the end result of chronic inflammatory reactions induced by trauma and can affect almost every organ system. Although most individual fibrotic diseases have a low incidence, fibrotic tissue responses are highly prevalent in chronic diseases such as asthma, chronic obstructive pulmonary disease (COPD), atherosclerosis, chronic inflammatory bowel diseases and heart failure after cardiac hypertrophy.

The cells that trigger fibrosis are activated fibroblasts (myofibroblasts) that express α-smooth muscle actin (α-SMA) and also have a higher expression of fibro-collagen (type I, III, V, and VI) and extra cellular matrix macromolecules. The differentiation of fibroblasts into myofibroblasts is produced by Transforming growth factor β (TGF-β), a major regulator of wound repair. Tissue responses to TGF-β are mediated through canonical (Smad-dependent) and Smad-independent intracellular signal transduction mechanisms. It was shown that overexpression of active TGF-β in animals produced fibrosis in the lungs, kidneys, and other organs.

The transcription factor, Early growth response-1 (EGR-1) is a novel Smad-independent mediator of TGF-β signaling that has been shown to play a role in the production of fibrosis. Stress signals, such as UV, hypoxia, and mechanical shear activate Egr-1 gene transcription in various cell types, including mouse and human fibroblasts and human fibrosarcoma cells. Cigarette smoke triggers chemokine release in primary human pulmonary fibroblasts in an ERK1/2 MAPK dependent manner. The Egr-1 gene is poorly expressed in the normal blood vessel wall but, when induced, can drive the expression of key growth factor and pro-inflammatory genes (e.g., PDGF-β, intercellular adhesion molecule-1 [ICAM-1], vascular cell adhesion molecule-1 [VCAM-1]). Egr-1 expression is increased in renal tubular cells in patients with renal failure. Mice deficient in Egr-1 were used to demonstrate that the gene promotes renal inflammation and fibrosis.

The transcription factor, Nuclear factor kappa-light-chain-enhancer of activated B cells (NF-κB) regulates the expression of a large number of genes in response to infection, inflammation, and other endogenous and exogenous stressors. NF-kB is normally inactive under resting conditions because it is bound to inhibitors of kB (IkB) (e.g., IkBa). NF-kB activation is triggered in response to a wide variety of stimuli and by activation of TNFα, Toll-like and T-cell receptors. Prolonged NF-κB activation has been described in chronic inflammatory lung diseases such as asthma, chronic obstructive pulmonary disease, and cystic fibrosis.

TNFaIP3 (A20) is an inducible, broadly expressed cytoplasmic protein. It terminates the activation of NFκB in response to stimulation by LPS, IL1β, TNFα, IL6 or CD40 and prevents NFκB-dependent upregulation of nucleotide-binding oligomerization domain-(NOD)-LRR and pyrin domain inflammasome (NLRP3) and conversion of pro-IL1β to mature IL1β through binding of its A20-like zinc finger domain to ubiquitin chains. It also blocks IKKα/β activation by the upstream kinase, Tak1. A body of research highlights A20 and the A20 “ubiquitin editing complex” as a potential therapeutic target for modulating chronic inflammatory diseases of the airways that are largely associated with exaggerated NF-kB activity.

### Lung Fibrosis

Idiopathic pulmonary fibrosis (IPF) is a chronic, interstitial lung disease of unknown etiology, characterized by thickened fibrotic alveolar walls. This results in impaired gas transfer, restricted ventilation and respiratory failure. The prevalence of IPF rises dramatically with age and is present in an estimated 0.2% of persons aged more than 75. The 5-year survival rate of patients with IPF is 20 to 40%, while the median survival ranges from 2 to 5 years from the time of diagnosis. The development of IPF is often preceded by acute lung inflammation caused by viral and bacterial infections, ionizing radiation, chemotherapy, air irritants and pollutants. Several factors, including age, genetic susceptibility, and environmental agents, are known to contribute to lung fibrosis.

Fibrosis in IPF was initially believed to be the result of chronic inflammation but studies demonstrated a lack of benefit with immunosuppressant agents, including corticosteroids, with or without immune modulator therapy, Interferon gamma 1b or Etanercept, that binds TNFα. However, the conclusion reached after a thorough examination of their effect was that these agents produced more harm than benefit, so the continued systemic use of these medications cannot be recommended.

### Skin Fibrosis

Skin fibrosis is characterized by marked thickening of the dermis with massive accumulation of dense collagen, causing epidermal atrophy and flattening of the epithelial extensions that project into the underlying connective tissue in both skin and mucous membranes. Early-stage skin biopsies reveal dermal edema and sometimes perivascular cellular infiltrates composed of T lymphocytes and monocytes

### Ladostigil

Ladostigil (6-(N-ethyl, N-methyl carbamyloxy)-N propargyl-1 (R)-aminoindan hemitartrate, was originally designed as an acetylcholinesterase (AChE) and brain-selective monoamine oxidase (MAO) inhibitor. At 20-fold lower concentrations than those inhibiting either enzyme, ladostigil prevented oxidative stress in cells by reducing the fall in the mitochondrial potential induced by Sin 1. Oxidative stress is a condition in which an imbalance exists between factors that promote oxidation and antioxidant defenses. It is involved in the pathogenesis of a variety of chronic diseases like cancer, atherosclerosis, coronary heart disease, lung fibrosis and Alzheimer's disease. Oxidative stress increases the expression of Nrf2, an antioxidant response regulator that activates transcription and expression of protective genes (Ma, 2013). At even lower concentrations than those reducing oxidative stress, ladostigil decreased gene expression of TNFα, IL-6 and IL1β in primary mouse microglial cultures activated by lipopolysaccharide (LPS) in association with a decrease in the nuclear translocation of NFκB.

### AN1284

AN1284 (3-(indolin-1-yl)-N-isopropylpropan-1-amine 2HCl) inhibited cytotoxicity resulting from oxidative stress and reduced the release of pro-inflammatory cytokines in LPS-activated macrophages. It acts by inhibiting phosphorylation of MAPK p38 and nuclear translocation of Activator protein-1. Chronic administration of AN1284 for two months by micro-osmotic pumps at the rate of 1 mg/kg/day, attenuated steatosis, lipogenesis, and fibrosis in mice with pre-existing non-alcoholic steatohepatitis (NASH) by activation of the Aryl hydrocarbon receptor (AhR). In a mouse model of lung fibrosis, activation of this receptor by another agent attenuated lung fibrosis. On the other hand, others have shown that activation of AhR in various tissues can increase fibrosis and worsen chronic obstructive pulmonary disease by increasing levels of EGR-1 protein.

There is still a great need for safe and effective compounds for use in treating fibrotic-related condition(s).

### SUMMARY OF THE INVENTION

According to the first aspect, there is provided a method of treating or preventing a fibrotic-related condition in a subject in need thereof, the method comprising administering to the subject a therapeutically effective amount of an antioxidant capable of: increasing expression level of tumor necrosis factor alpha induced protein 3 (TNFαIP3), reducing expression level of early growth response protein (EGR) 1 and early EGR2, or any combination thereof, thereby treating or preventing fibrotic-related condition in the subject.

According to another aspect, there is provided a pharmaceutical composition comprising a therapeutically effective amount of an antioxidant capable of: increasing expression level of tumor necrosis factor alpha induced protein 3 (TNFαIP3), reducing expression level of early growth response protein (EGR) 1 and early EGR2, or any combination thereof, for use in the treatment or prevention of a fibrotic-related condition in a subject in need thereof.

In some embodiments, the antioxidant comprises ladostigil, AN1284, or a combination thereof.

In some embodiments, the fibrotic-related condition comprises lung fibrosis, skin sclerosis, or both.

In some embodiments, the administering comprises administering a pharmaceutical composition comprising a therapeutically effective amount of the antioxidant, and a pharmaceutically acceptable carrier.

In some embodiments, the treating comprises reducing level of transforming growth factor beta (TGFβ) in the subject. In some embodiments, the treating comprises reducing the number of cells expressing TGFβ in the subject. In some embodiments, the treating comprises reducing the number of cells expressing TGFβ in the lung, the skin, or both, of the subject. In some embodiments, the treating comprises reducing surface area of cells expressing TGFβ in the subject. In some embodiments, surface is skin surface. In some embodiments, skin surface comprises surface on the epidermis, dermis, hypodermis, or any combination thereof, of the skin.

In some embodiments, expressing comprises overexpressing. In some embodiments, overexpressing is compared to a control. In some embodiments, a control comprises or refers to a basal, benchmark, naïve, standard, average, or any combination thereof, of TGFβ expression levels. In some embodiments, a control comprises TGFβ expression levels of a fibroblast obtained or derived from a control subject.

In some embodiments, the treating comprises reducing the thickness of the dermis of the subject. In some embodiments, the treating comprises increasing the thickness of the hypodermis of the subject. In some embodiments, the treating comprises reducing the thickness of the dermis and increasing the thickness of the hypodermis of the subject.

In some embodiments, the treating comprises increasing weight gain in the subject.

In some embodiments, the treating comprises reducing weight loss in the subject.

In some embodiments, any one of reducing, increasing, or both, is compared to a control. In some embodiments, a control comprises a control subject. In some embodiments, a control subject is a non-treated subject. In some embodiments, a control comprises a subject not being treated according to the method of the invention, or with the composition for use of the invention.

In some embodiments, the control subject is the subject treated according to the method of the invention or with the composition for use of the invention, before being treated according to the method of the invention or with the composition for use of the invention.

In some embodiments, the treating comprises maintaining levels of TNFα, Interleukin (IL) 6, or both, in the subject.

In some embodiments, the pharmaceutical composition further comprises a pharmaceutically acceptable carrier, excipient, or adjuvant.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

FIG. 1 includes a vertical bar graph showing dose-related reduction in release of tumor necrosis factor alpha (TNFα), Interleukin (IL) 6, and IL1β from microglia activated by 2′-3′-O-(4-benzoyl benzoyl) adenosine 5′-triphosphate (BzATP)/lipopolysaccharides (LPS). Ladostigil or budesonide were added 2 hrs before BzATP and LPS in the presence of 0.1% BSA. ANOVA for TNFα; F_{5,275}=85.4, p<0.0001; IL6; F_{5,282}=141.7, p<0.0001; IL1β; F_{4,73}=18.14, p<0.0001. All concentrations of ladostigil and budesonide tested reduced the 3 cytokines significantly ** p<0.01. Significantly different from value for TNFα, #p<0.05.

FIGS. 2A-2B include volcano plots showing that ladostigil treatment alters gene expression in BzATP/LPS-activated microglia. (2A) Volcano plots showing the log-fold-change vs. −log 10(q-value) as calculated by edgeR) and differential expression analysis in ladostigil treated microglia compared to untreated cells. (2B) Volcano plots showing the log-fold-change vs. −log −log 10(q-value) of ladostigil treated microglia, 8 hrs after addition of BzATP/LPS. Blue dots indicate genes that are downregulated and red, those up regulated by ladostigil. Cut-off value, ±0.5.

FIGS. 3A-3B include horizontal and vertical bar graphs showing Genes altered by ladostigil treatment. (3A) Bars show the change caused by ladostigil in gene expression in microglia after BzATP/LPS-induced activation. Bars to the right show genes that were up-regulated and those to the left, downregulated. Cut-off value, ±0.5. (3B) Selected, differentially expressed genes after BzATP/LPS treatment, with or without ladostigil. FDR=False Discovery Rate. PC=Principal component. Significantly different from control unstimulated, *** p<0.001; significant effect of ladostigil, #p<0.05; ##p<0.01 ###p<0.001.

FIGS. 4A-4B include graphs showing that control mice of both sexes showed an increase of 2-4% in body weight. Bleomycin caused a similar weight loss in mice of both sexes. Ladostigil significantly reduced weight loss in males (4A) only at a dose of 10 mg/kg, but in females at doses of 5 and 10 mg/kg (4B). Significantly different of bleomycin ^{a} p<0.05; ^{b} p<0.01, ^{c} p<0.001. In 5A scale bar=100 μm.

FIGS. 5A-5C include micrographs, and vertical bar graphs showing the effect of ladostigil on: collagen content (fibrosis; 5A), fibrosis score (5B), and collagen levels (hydroxyproline; 5C). Data represent mean #STD from 7-10 mice. Significantly different from saline, ** p<0.01; *** p<0.01. Significantly different of bleomycin #p<0.05; ##p<0.01, ###p<0.001. In 5A scale bar=100 μm.

FIG. 6 includes vertical bar graphs showing the lack of effect of ladostigil on cytokines in bronchial fluid. Significantly different from saline * p<0.05; significantly different from bleomycin, ##p<0.01.

FIGS. 7A-7B include histological micrographs and a graph showing the effect of bleomycin and ladostigil on TGFβ in the lung. (7A) Sections of lung from mice treated with: negative control (Left), bleomycin alone (Middle), and bleomycin+ladostigil (Right). Cells containing TGFβ (“positive cells”) are stained in brown (representative positive cells are indicated by arrowheads). Calibration line=400 μm. (7B) A graph showing the average number of cells containing TGFβ per cell from 3-6 cells per mouse. Significantly different from saline, * p<0.05. Significantly different from bleomycin, #p<0.05.

FIG. 8 includes a showing that control mice and those given ladostigil showed an increase of 15% in body weight on days 24-27. Bleomycin caused a smaller increase in weight on those days than saline. Weight gain in mice administered with ladostigil was greater than in those on bleomycin on days 18, 21, 24 and 27. Significantly different from bleomycin ^{a} p<0.05; ^{b} p<0.01.

FIGS. 9A-9B include micrographs and a graph showing the effect of bleomycin and ladostigil on dermal and hypodermal thickness. (9A) Histological sections of skin from mice treated with: negative control (Left), bleomycin alone (Middle), and bleomycin+ladostigil (Right). (9B) A graph showing the reduction by ladostigil of dermal thickness and increase in hypodermal thickness. Significantly different from bleomycin #p<0.05.

FIG. 10 include a graph showing the effect of bleomycin and ladostigil on the area of each skin section showing TGFβ-containing cells. Significantly different from saline * p<0.05; significantly different from bleomycin, #p<0.05.

FIG. 11 includes a graph showing the effect of AN1284 of collagen (fibrosis score). Significantly different from saline ** p<0.01; significantly different from bleomycin, ##p<0.01.

FIG. 12 includes a graph showing the lack of effect of AN1284 on pro-inflammatory cytokines in bronchial fluid. Significantly different from bleomycin, ** p<0.01.

### DETAILED DESCRIPTION OF THE INVENTION

According to the first aspect, there is provided a method of treating or preventing a fibrotic-related condition in a subject in need thereof.

In some embodiments, the method comprises administering to the subject a therapeutically effective amount of an antioxidant. In some embodiments, the antioxidant comprises an antioxidant capable of increasing expression level of TNFα induced protein 3 (TNFαIP3), reducing the expression level of EGR1, EGR2, or both, or any combination thereof.

As used herein, the term “antioxidant” refers to any compound capable of scavenging, neutralizing, or both, free radicals, such as oxygen radicals.

Types of antioxidants, as well as methods and means for determining antioxidant activity are common and would be apparent to one of ordinary skill in the art.

In some embodiments, the antioxidant is or comprises ladostigil or a pharmaceutically active salt thereof, 3-(indolin-1-yl)-N-isopropylpropan-1-amine 2HCl (AN1284) or a pharmaceutically active salt thereof, or any combination thereof.

In some embodiments, increasing or decreasing is compared to a control. In some embodiments, a control comprises a healthy control. In some embodiments, a control comprises a control antioxidant. In some embodiments, a control antioxidant does not: increase expression level of TNFαIP3, reduce the expression level of EGR1, EGR2, or both, or any combination thereof. In some embodiments, control is a subject or a cell not treated or administered with an antioxidant being capable of increasing expression level of TNFα induced protein 3 (TNFαIP3), reducing the expression level of EGR1, EGR2, or both, or any combination thereof.

As used herein, the term “fibrotic-related condition” encompasses any disease, disorder, or a symptom associated therewith, comprising, characterized by, induced by, propagated by, or any combination thereof, tissue fibrosis.

As used herein, the term “fibrosis” refers to conditions in which excessive connective tissue is present and, therefore, to conditions in which there is increased collagen deposition compared to healthy individuals. The term “fibrosis” also includes reference to conditions in which there is increased deposition of other extracellular matrix (“ECM”).

In some embodiments, the fibrotic-related condition comprises a fibrotic-related disease. In some embodiments, the fibrotic-related condition comprises a manifestation of fibrosis. In some embodiments, the manifestation of fibrosis is at least one symptom associated with a non-fibrotic disease. In some embodiments, the manifestation of fibrosis is a symptom associated with post-surgery.

In some embodiments, treating or treatment of fibrotic-related condition does not include or excludes treating or treatment of inflammation.

In some embodiments, the method of the invention is not intended to treat or prevent inflammation in a subject.

In some embodiments, a fibrotic-related condition comprises fibrosis in a tissue selected from: lung, liver, heart, retinal, mediastinal, retroperitoneal cavity, bone marrow, skin, or any combination thereof.

In some embodiments, a fibrotic-related condition comprises the manifestation of fibrosis in a tissue selected from: lung, liver, heart, retinal, mediastinal, retroperitoneal cavity, bone marrow, skin, or any combination thereof.

In some embodiments, a fibrotic-related condition comprises or is selected from: lung fibrosis or pulmonary fibrosis, liver fibrosis, heart fibrosis, mediastinal fibrosis, retroperitoneal cavity fibrosis, bone marrow fibrosis, skin fibrosis, Scleroderma or systemic sclerosis, or any combination thereof.

In some embodiments, a fibrotic-related condition comprises or is lung fibrosis.

In some embodiments, the administering comprises administering a pharmaceutical composition comprising a therapeutically effective amount of ladostigil.

In some embodiments, the pharmaceutical composition further comprises pharmaceutically acceptable carrier, excipient, or adjuvant.

In some embodiments, the treating or preventing does not include reducing expression, secretion, amount, abundance, or any combination thereof, of at least one pro-inflammatory gene or a protein product thereof. In some embodiments, the at least one pro-inflammatory gene encodes a cytokine (e.g., the protein product).

In some embodiments, the treating comprises reducing expression, secretion, amount, abundance, or any combination thereof, of the transforming growth factor beta (TGFβ).

In some embodiments, reducing expression, secretion, amount, abundance, or any combination thereof, of the transforming growth factor beta (TGFβ) is in at least one cell of the subject. In some embodiments, the cell comprises or is a fibroblast.

In some embodiments, expression comprises gene and/or RNA expression level. In some embodiments, expression comprises protein expression level.

Methods and means for determining gene and/or RNA and/or protein level, e.g., of TNFαIP3, EGR1, EGR2, or any combination thereof, including abundance, location, temporal expression, special expression, etc., are common and would be apparent to one of ordinary skill in the art. Non-limiting examples for methods of expression determination include, but are not limited to, PCR, RT-PCR, quantitative PCR, western blot, dot blot, MS/MS, next generation sequencing, and others.

In some embodiments, the method further comprises determining expression level of TNFα, IL-6, TGFβ, or any combination thereof. In some embodiments, the method further comprises determining expression level of TGFβ. In some embodiments, a determined TGFβ level being reduced compared to non-treated control is indicative of the subject being responsive to the treatment. In some embodiments, a determined TGFβ level being maintained or essentially similar to non-treated control is indicative of the subject not being responsive or being resistant to the treatment. In some embodiments, TGFβ is or comprises TGFβ1.

In some embodiments, a non-responsive or resistant subject it to be withdrawn from the treatment. In some embodiments, withdrawing a subject being non-responsive or resistant to the treatment.

In some embodiments, essentially similar is 80-99%, 85-99%, 90-99%, 95-99% or 97-99% of a non-treated control. Each possibility represents a separate embodiment of the invention.

In some embodiments, essentially similar is identical. In some embodiments, identical is 100% identical to a non-treated control.

In some embodiments, a non-treated control is a non-treated control subject (or any sample obtained or derived therefrom). In some embodiments, a non-treated control is a subject (or any sample obtained or derived therefrom) before being treated according to the method or use of the invention.

In some embodiments, treating or preventing is treating or preventing the condition or disease in the subject.

In some embodiments, the at least one pro-inflammatory gene or a protein product thereof is selected from: tumor necrosis factor alpha (TNFα) or interleukin (IL) 6.

In some embodiments, a control is a control subject or a sample obtained or derived therefrom.

In some embodiments, a control subject is not administered with ladostigil. In some embodiments, a control subject is not administered with an effective amount of ladostigil.

In some embodiments, a control subject is administered with bleomycin.

According to another aspect, there is provided a pharmaceutical composition comprising a therapeutically effective amount of ladostigil, for use in the treatment or prevention of a fibrotic-related condition, in a subject in need thereof.

As used herein, the terms “treatment” or “treating” of a disease, disorder, or condition (e.g., fibrotic-related condition) encompasses alleviation of at least one symptom thereof, a reduction in the severity thereof, or inhibition of the progression thereof. Treatment need not mean that the disease, disorder, or condition is totally cured. To be an effective treatment, a useful composition or method herein needs only to reduce the severity of a disease, disorder, or condition, reduce the severity of symptoms associated therewith, or provide improvement to a patient or subject's quality of life.

In some embodiments, treating or preventing is treating. In some embodiments, treating or preventing is preventing. In some embodiments, treating comprises preventing. In some embodiments, treating comprises decreasing volume, weight, or both, of a fibrotic tissue. In some embodiments, decreasing comprises a statistically significant change. In some embodiments, increasing comprises a statistically significant change. In some embodiments, a statistically significant change is at least a 10, 15, 20, 25, 30, 35, 40, 45, 50, 55, 60, 65, 70, 75, 80, 85, 90, 92, 95, 97, 99, 100, 150, 200, 250, 300, 350, 400, 450 or 500% change, or any value and range in between. Each possibility represents a separate embodiment of the invention. In some embodiments, treating comprises preventing increase or growth (e.g., volume, weight, or both) of a fibrotic tissue in the subject.

As used herein, the terms “administering”, “administration” and like terms refer to any method which, in sound medical practice, delivers a composition containing an active agent to a subject in such a manner as to provide a therapeutic effect. One aspect of the present subject matter provides for oral administration of a therapeutically effective amount of ladostigil to a subject in need thereof. Other suitable routes of administration can include intravenous, parenteral, subcutaneous, intramuscular, or intraperitoneal. In some embodiments, administering is oral administration. In some embodiments, administering is pulmonary administration. In some embodiments, administering is intranasal administration. In some embodiments, administering is intra-tracheal administration.

The dosage administered will be dependent upon the age, health, and weight of the recipient, kind of concurrent treatment, if any, frequency of treatment, and the nature of the effect desired.

In some embodiments, the ladostigil is a therapeutically effective amount of ladostigil. In some embodiments, a therapeutically effective amount is a therapeutically effective dose. The term “therapeutically effective amount” refers to an amount of a drug effective to treat a disease or disorder (e.g., cancer) in a mammal. The term “a therapeutically effective amount” refers to an amount effective, at dosages and for periods of time necessary, to achieve the desired therapeutic or prophylactic result (e.g., treating a fibrotic-related condition). The exact dosage form and regimen would be determined by the physician according to the patient's condition. In some embodiments, ladostigil is administered at a daily dose of 1 to 25 mg. In some embodiments, ladostigil is administered at a daily dose of 10 to 25 mg.

In some embodiments, the methods of the invention are affected by administering to a subject in need thereof ladostigil or a pharmaceutically active salt thereof. In some embodiments, the ladostigil is a salt of ladostigil. Salts of ladostigil include the 1/2 L-tartrate salt of ladostigil. This tartrate salt of ladostigil, ladostigil tartrate-6-(N-ethyl, N-methyl carbamyloxy)-N-propargyl-1 (R)-aminoindan, tartaric acid (2:1) abbreviated as [(R)-CPAI] tartrate and also referred to as ladostigil tartrate, has CAS registry number 209394-46-7 and may be used as the active ingredient of ladostigil tablets.

In some embodiments, the ladostigil is a crystalline form of ladostigil tartrate. U.S. Patent Application Publication Nos. 20060189819, 20070088082 and 20070093549, which are incorporated herein by reference in their entirety, disclose crystalline forms of ladostigil tartrate and methods of producing the same. U.S. Pat. Nos. 7,375,249 and 7,476,757, and U.S. Patent Application Publication No. 20060199974, which are incorporated herein by reference in their entirety, disclose synthesis of enantiomeric indanylamine derivatives including ladostigil. U.S. Pat. No. 7,491,847 and U.S. Patent Application Publication No. 20070112217, which are incorporated herein by reference in their entirety, disclose methods for isolating propargylated aminoindans.

U.S. Patent Application Publication No. 20060189685, which is incorporated herein by reference in its entirety, discloses formulations comprising ladostigil.

In some embodiments, the composition further comprises a pharmaceutically acceptable carrier, excipient, or adjuvant. As used herein, the term “carrier”, “excipient”, or “adjuvant” refers to any component of a pharmaceutical composition that is not the active agent. As used herein, the term “pharmaceutically acceptable carrier” refers to non-toxic, inert solid, semi-solid liquid filler, diluent, encapsulating material, formulation auxiliary of any type, or simply a sterile aqueous medium, such as saline. Some examples of the materials that can serve as pharmaceutically acceptable carriers are sugars, such as lactose, glucose and sucrose, starches such as corn starch and potato starch, cellulose and its derivatives such as sodium carboxymethyl cellulose, ethyl cellulose and cellulose acetate; powdered tragacanth; malt, gelatin, talc; excipients such as cocoa butter and suppository waxes; oils such as peanut oil, cottonseed oil, safflower oil, sesame oil, olive oil, corn oil and soybean oil; glycols, such as propylene glycol, polyols such as glycerin, sorbitol, mannitol and polyethylene glycol; esters such as ethyl oleate and ethyl laurate, agar; buffering agents such as magnesium hydroxide and aluminum hydroxide; alginic acid; pyrogen-free water; isotonic saline, Ringer's solution; ethyl alcohol and phosphate buffer solutions, as well as other non-toxic compatible substances used in pharmaceutical formulations. Some non-limiting examples of substances which can serve as a carrier herein include sugar, starch, cellulose and its derivatives, powered tragacanth, malt, gelatin, talc, stearic acid, magnesium stearate, calcium sulfate, vegetable oils, polyols, alginic acid, pyrogen-free water, isotonic saline, phosphate buffer solutions, cocoa butter (suppository base), emulsifier as well as other non-toxic pharmaceutically compatible substances used in other pharmaceutical formulations. Wetting agents and lubricants such as sodium lauryl sulfate, as well as coloring agents, flavoring agents, excipients, stabilizers, antioxidants, and preservatives may also be present. Any non-toxic, inert, and effective carrier may be used to formulate the compositions contemplated herein. Suitable pharmaceutically acceptable carriers, excipients, and diluents in this regard are well known to those of skill in the art, such as those described in The Merck Index, Thirteenth Edition, Budavari et al., Eds., Merck & Co., Inc., Rahway, N.J. (2001); the CTFA (Cosmetic, Toiletry, and Fragrance Association) International Cosmetic Ingredient Dictionary and Handbook, Tenth Edition (2004); and the “Inactive Ingredient Guide,” U.S. Food and Drug Administration (FDA) Center for Drug Evaluation and Research (CDER) Office of Management, the contents of all of which are hereby incorporated by reference in their entirety. Examples of pharmaceutically acceptable excipients, carriers, and diluents useful in the present compositions include distilled water, physiological saline, Ringer's solution, dextrose solution, Hank's solution, and DMSO. These additional inactive components, as well as effective formulations and administration procedures, are well known in the art and are described in standard textbooks, such as Goodman and Gillman's: The Pharmacological Bases of Therapeutics, 8th Ed., Gilman et al. Eds. Pergamon Press (1990); Remington's Pharmaceutical Sciences, 18th Ed., Mack Publishing Co., Easton, Pa. (1990); and Remington: The Science and Practice of Pharmacy, 21st Ed., Lippincott Williams & Wilkins, Philadelphia, Pa., (2005), each of which is incorporated by reference herein in its entirety. The presently described composition may also be contained in artificially created structures such as liposomes, ISCOMS, slow-releasing particles, and other vehicles which increase the half-life of the peptides or polypeptides in serum. Liposomes include emulsions, foams, micelles, insoluble monolayers, liquid crystals, phospholipid dispersions, lamellar layers, and the like. Liposomes for use with the presently described peptides are formed from standard vesicle-forming lipids which generally include neutral and negatively charged phospholipids and sterol(s), such as cholesterol. The selection of lipids is generally determined by considerations such as liposome size and stability in the blood. A variety of methods are available for preparing liposomes as reviewed, for example, by Coligan, J. E. et al, Current Protocols in Protein Science, 1999, John Wiley & Sons, Inc., New York, and see also U.S. Pat. Nos. 4,235,871, 4,501,728, 4,837,028, and 5,019,369.

The carrier may comprise, in total, from about 0.1% to about 99.99999% by weight of the pharmaceutical compositions presented herein. In some embodiments, the composition consists of ladostigil. In some embodiments, the composition consists essentially of ladostigil. In some embodiments, the composition comprises ladostigil as the only therapeutic agent. In some embodiments, the therapeutic agent is a therapeutic anti-fibrotic/fibrosis agent. In some embodiments, the composition is devoid of another therapeutic agent other than ladostigil. In some embodiments, the ladostigil is essentially pure ladostigil. In some embodiments, the composition comprises an active agent and a carrier, wherein the active agent consists essentially of ladostigil.

As used herein, the term “consists or consisting essentially of” denotes that a given compound or substance constitutes the vast majority of the active ingredient's portion or fraction of the composition.

In some embodiments, consists essentially of means that ladostigil constitutes at least 95%, at least 98%, at least 99%, or at least 99.9% by any one of: weight, mole, or molarity, of the composition, or any value and range there between. Each possibility represents a separate embodiment of the invention.

In some embodiments, the subject is administered ladostigil as a monotherapy. In some embodiments, the subject is administered ladostigil as part of a combination therapy. In some embodiments, the method further comprises administering at least one other anti-fibrosis therapy.

According to another aspect, there is provided a pharmaceutical composition comprising ladostigil in a unit dose.

In some embodiments, the pharmaceutical composition comprises a low dose of ladostigil. In some embodiments, a low dose of ladostigil comprises ladostigil in an amount effective for inhibiting, reducing, alleviating, treating, or any combination thereof, fibrosis.

In some embodiments, a low dose of ladostigil comprises ladostigil given at a time that does not show a reduction in inflammation.

In some embodiments, a low dose of ladostigil comprises ladostigil in an amount effective for inhibiting, reducing, alleviating, treating, or any combination thereof, fibrosis.

As used herein, a “unit dose” is a discrete amount of the pharmaceutical composition comprising a predetermined amount of the active ingredient (e.g., ladostigil). The amount of the active ingredient is generally equal to the dosage of the active ingredient which would be administered to a subject and/or a convenient fraction of such a dosage such as, for example, one-half or one-third of such a dose.

In some embodiments, a unit dose comprises 1 mg to 25 mg ladostigil per Kg of a subject body weight. In some embodiments, a unit dose comprises: at least 1 mg, 2 mg, 3 mg, 4 mg, 5 mg, 8 mg, 10 mg, 12 mg, 15 mg, 20 mg, or 25 mg ladostigil per Kg of a subject body weight, including any value and range in between.

In some embodiments, a unit dose comprises ladostigil in an amount insufficient for inhibiting or reducing the activity of acetylcholine esterase (AChE), monoamine oxidase (MAO), or both, and insufficient for inhibiting or reducing the activity of a pro-inflammatory cytokine, as disclosed herein, e.g., TNFα, IL6, etc.

In some embodiments, a unit dose comprises ladostigil in an amount sufficient for inhibiting or reducing the activity of acetylcholine esterase (AChE), monoamine oxidase (MAO), or both, by no more than 1%, 2%, 3%, 4%, 5%, 6%, 7%, 8%, 9% 10%, or any value and range in between, and insufficient for inhibiting or reducing the activity of a pro-inflammatory cytokine, as disclosed herein, e.g., TNFα, IL6, etc. Each possibility represents a separate embodiment of the invention.

In some embodiments, a unit dose comprises ladostigil in an amount sufficient for reducing or inhibiting fibrosis, maintaining the activity of acetylcholine esterase (AChE), monoamine oxidase (MAO), or both, and maintaining the activity of a pro-inflammatory cytokine, as disclosed herein, e.g., TNFα, IL6, etc.

In some embodiments, a unit dose comprises ladostigil in an amount sufficient for reducing or inhibiting fibrosis, insufficient for inhibiting or reducing the activity of acetylcholine esterase (AChE), monoamine oxidase (MAO), or both, and insufficient for inhibiting or reducing the activity of a pro-inflammatory cytokine, as disclosed herein, e.g., TNFα, IL6, etc.

In some embodiments, an amount sufficient for reducing or inhibiting fibrosis is insufficient for inhibiting or reducing the activity of acetylcholine esterase (AChE), monoamine oxidase (MAO), or both, insufficient for inhibiting or reducing the activity of a pro-inflammatory cytokine, as disclosed herein, e.g., TNFα, IL6, etc., or any combination thereof.

In some embodiments, an amount sufficient for reducing or inhibiting fibrosis is sufficient for inhibiting or reducing the activity of acetylcholine esterase (AChE), monoamine oxidase (MAO), or both, by no more than 1%, 2%, 3%, 4%, 5%, 6%, 7%, 8%, 9% 10%, or any value and range in between, insufficient for inhibiting or reducing the activity of a pro-inflammatory cytokine, as disclosed herein, e.g., TNFα, IL6, etc., or any combination thereof.

As used herein, the term “about” when combined with a value refers to plus and minus 10% of the reference value. For example, a length of about 1,000 nanometers (nm) refers to a length of 1,000 nm±100 nm.

It is noted that as used herein and in the appended claims, the singular forms “a,” “an,” and “the” include plural referents unless the context clearly dictates otherwise. Thus, for example, reference to “a polynucleotide” includes a plurality of such polynucleotides and reference to “the polypeptide” includes reference to one or more polypeptides and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

In those instances, where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

### EXAMPLES

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells-A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, CT (1994); Mishell and Shiigi (eds), “Strategies for Protein Purification and Characterization-A Laboratory Course Manual” CSHL Press (1996); all of which are incorporated by reference. Other general references are provided throughout this document.

### Materials and Methods

### Preparation of Microglial Cultures

Experiments were performed on primary microglial cells isolated from brains of neonatal Balb/C mice as previously described (Reichert and Rotshenker, 2003). The brains were stripped of their meninges and enzymatically dissociated. Cells were plated on Poly-L-lysine coated flasks for one week, re-plated for 1- to 2-hr on bacteriological plates and non-adherent cells were washed away. Microglia were propagated by incubation in 20% of medium, conditioned by the L-cell line that produces mouse-CSF. They were identified by morphology and positive immune-reactivity to P2y12, F4/80, complement receptor-3 and Galectin-3/MAC-2 (Reichert and Rotshenker 2003; Gitik et al. 2011).

### Measurement of Cytokines

Cytokines were measured as previously described (Shamash et al. 2002) using ELISA Max deluxe set (Biolegend, CA, USA) for secreted TNFα and IL-6 proteins and ELISA DuoSet (R&D systems, UK) for secreted and cell associated IL-1β, according to manufacturer's instructions.

The effect of ladostigil at concentrations of 1×10⁻¹³ M to 1×10⁻⁹ M on the secretion of cytokine proteins was assessed by adding it to microglia for 2 hr before LPS (0.75 μg/ml) and BzATP (400 μM). Other microglia were treated similarly with the steroid, budesonide as a reference standard at concentrations of 1× 10⁻¹³ to 1× 10⁻¹¹ M. Measurements of cytokine secretion were performed 8 hrs after addition of LPS and BzATP. Since the levels of secreted IL-1β were still low, samples were concentrated 2- to 4-fold by an Amicon ultra-centrifugal filter device (Merck-Millipore, Ireland). ELISA was used to quantify the levels of cytokine proteins. Protein content in the microglia lysate was measured by Bradford and levels of cytokines were calculated and presented as pg/μg of microglial protein.

### RNA-Seq of Microglia

Ladostigil (1×10⁻¹⁰ M) was added to microglia for 2 hrs before BzATP/LPS. Cells were harvested before and 8 hr after addition of BzATP/LPS. Total RNA was extracted using the RNeasy Plus Universal Mini Kit (QIAGEN) according to the manufacturer's protocol. Total RNA samples (1 μg RNA) were enriched for mRNAs by pull-down of poly A. Libraries were prepared using the KAPA Stranded mRNA-Seq Kit according to the manufacturer's protocol and sequenced using Illumina NextSeq 500 to generate 85 bp single-end reads (total 25-30 million reads per sample) as described in Zohar et al. (2021).

### Bioinformatic Analysis

Next-generation sequencing data underwent quality control using FastQC. They were then preprocessed using Trimmomatic (Bolger et al. 2014) and aligned to the reference genome GRCm38 with the STAR aligner (Dobin et al. 2013) using default parameters. Genomic loci were annotated using GENCODE version M25 (Frankish et al. 2019). Genes with low expression were filtered out of the dataset by setting a threshold of a minimum of 2 counts-per-million in three samples.

### Gene Module Classification

Pair-wise differential analyses were performed between all three BzATP/LPS time points and genes with an FDR <0.01 were considered. Only genes with an absolute log fold-change of >0.5 between two consecutive time points were labeled as up- or down-regulated.

### Effect of Ladostigil on Lung Fibrosis

The animal model of bleomycin-induced lung fibrosis is widely used to characterize the potential inhibitory effect of newly developed drugs (Distler et al. 2019). Experiments were performed in C57BL/6JOlaHsd male and female mice aged 12-13 weeks. Male mice weighed 23-27.3 g and female mice, 19.4-21.1 g. They were housed under pathogen-free conditions, up to 6 per cage, in IVC cages under a controlled temperature of 22-24° C., humidity and alternating 12-h light/dark cycles and provided with food and water ad libitum. In order to minimize the stress of daily injection, ladostigil (5 mg/Kg/day) was administered by micro-osmotic pumps, implanted subcutaneously under ketamine/xylazine anesthesia, 5 days before administration of bleomycin, and delivered for 3 weeks to mice of each sex. Saline (2-3/sex) and bleomycin controls were anesthetized as described above. Bleomycin solution (0.25 mg/kg in 50 μl) was instilled into the lumen of the trachea as shown in a video by Ehrentraut et al. (2019). Although intra-tracheal bleomycin does not replicate all the changes induced in humans with chronic lung disease it is able to induce molecular changes that are directly relevant to IPF (Peng et al. 2013). Mice were weighed daily and sacrificed on day 21. Bronchial fluid was collected as described in Ehrentraut et al. (2019). TNFα and IL-6 in the bronchial fluid lavage was measured by ELISA as described in the preceding section.

### Histopathological Analysis

Specimens taken from the left lung were embedded in paraffin and cut into 5 μm-thick slices. They were put on slides and paraffin was removed. They were rehydrated and stained with a solution of Sirius red (100 mg/100 ml of picric acid), rinsed in distilled water until clear, dehydrated and mounted in a resinous medium. The red stain (also called “Sirius red” stain) is one of the best understood histochemical techniques. It relies on the birefringent properties of collagen and is much more specific than the other common collagen stains, Pulmonary fibrosis was evaluated on a score of 0-4 on 15-20 different fields taken from each lung section, according to the area and intensity of fibrotic staining in the field, excluding that in blood vessels. Two independent scientists blinded to the treatment performed the analysis. For TGFβ1 staining, slides were deparaffinized and rehydrated. Antigen retrieval was performed in Tris-based buffer, pH 9.0. Endogenous peroxidase activity was quenched by incubation in 3% H2O2. The tissue was permeabilized with Triton X-100 0.1% in PBS. After blocking, sections were incubated with primary antibody: diluted rabbit recombinant monoclonal TGFβ1 antibody (Abcam ab215715) over night, at 4° C. Slices were further incubated with the secondary antibody: ImmPRESS®-HRP Goat Anti-Rabbit IgG Polymer Detection Kit, Peroxidase (Vector labs VE-MP-7451-15) at room temperature. For color development, sections were incubated in peroxidase substrate solution DAB, at room temperature until desired stain intensity developed. They were then counterstained with filtered hematoxylin and cosin, cleared and mounted.

### Induction of Skin Sclerosis by Bleomycin Treatment

Male C57BL/6 mice aged 8 weeks were housed under pathogen-free conditions at a controlled temperature of 22-24° C., humidity and alternating 12-h light/dark cycles and provided with food and water ad libitum. Skin sclerosis was induced as described by Ravanetti et al, (2020). A small incision was made in the skin at the back caudal to the scapulae and subcutaneous pocket was created into which an Alzet micro-osmotic pump was implanted, and the incision closed with two stiches and a surgical clip. The pumps contained bleomycin, (delivered at the rate of 60 U/kg body weight) or saline (control) and were removed 10 days after implantation. The mice were monitored daily until they recovered completely and weighed twice weekly from the beginning of the experiment.

### Effect of Ladostigil on Skin Sclerosis

Ladostigil (10 mg/kg/day) was administered in the drinking fluid to 10 mice, housed 2/cage, from 3 days before bleomycin pump installation until the end of the experiment. The fluid intake of the mice was measured daily. The dose of ladostigil was adjusted accordingly. Bleomycin alone, (10 mice) and saline controls (6 mice) received regular drinking fluid.

### Measurement of Skin Thickness.

Skin was harvested on the gluteal region to avoid dermal fibrosis artifact due to proximity to the site of implant. For histological assessment, the samples were dehydrated in a graded ethanol series, clarified in xylene, and paraffin embedded. They were mounted on slides and paraffin was removed. Sections of 5 μm thickness were cut with a rotary microtome and stained with hematoxylin and eosin (H&E)

### Measurement of Oxidative Stress in SH-SY5Y Cells

SH-SY5Y cells were cultured in F12 medium containing 10% fetal calf serum and 1:10 L-Alanyl-L-Glutamine. The cells are incubated at 37° C. and 5% CO₂, then rinsed with phosphate-buffered saline (PH=7.4) until they reached a growth state of more than 80% differentiation, and then 2 mL of trypsin-EDTA mixture was added to separate the attached cells before centrifugation at 112 g for 3 min. When the cells were more than 80% differentiated, 2.5×10⁵ cells/well were seeded in a 96-well plates and incubated at 37° C. for 24 h with increasing concentrations of ladostigil or AN1284. Cell viability was assessed by MTT.

Measurement of Cytokine Release from LPS Activated RAW 264.7 Cells

RAW 264.7 cells were seeded at a density of 1×10⁵ cells per well in 48-well culture plates and grown overnight in DMEM supplemented as indicated above. The wells were washed with PBS (pH 7.4). AN1284, prepared in sterile double distilled water and Budesonide (positive control) were added to give final concentrations ranging from 1× 10⁻¹³-1×10⁻⁷M in medium containing 2% FCS. The cells were incubated for 2 h at 37° C. prior to stimulation with LPS, 2.5 μg/mL from Escherichia coli (Sigma-Aldrich serotype 0111: B4). Supernatants were harvested after 8 hrs for measurement of TNF-α and 24 h for IL-6. TNF-α and IL-6 protein were detected by a commercial ELISA kit (Biolegend, San Diego, CA, USA) according to the manufacturer's instructions. Each concentration of each compound was tested in two-three separate experiments in 6 wells per concentration.

LPS also increases superoxide, a major reactive oxygen species. Nrf2 regulates the action of antioxidant proteins that can help protect against oxidative damage. A reduction in oxidative stress by compounds like AN1284 decreases Nrf2 because less is needed.

### Measurement of Proteins

RAW 264.7 cells were lysed in 0.1 ml of buffer containing 150 mM Tris-Cl (pH 6.8), 10% Glycerol, 0.6% SDS, Bromophenol Blue and 100 mM β-Mercaptoethanol. Lysates were collected and heated at 95° C. for 5 min, then centrifuged for 3 min at 2,240 g. Supernatants were used for gel separation. Protein concentration of the lysates was determined by using Coomassie Brilliant Blue staining. For western blot analysis 20-60 μg protein were loaded on 10% SDS-PAGE gel and electrophoresed. After blotting to a nitrocellulose membrane (Whatman), the blots were blocked for 1 h, at room temperature in TBS-T (25 mM Tris-HCl pH 7.4, 0.9% NaCl and 0.02% Tween-20) with 4% Difco skim milk. Molecular changes were assessed semi-quantitatively after incubation with primary antibodies for relevant proteins.

### Effect of AN1284 on Lung Fibrosis

Experiments were performed in C57BL/6JOlaHsd male mice aged 12-13 weeks as described for ladostigil. AN1284 (2.5 mg/kg/day) was administered by micro-osmotic pumps, implanted subcutaneously under ketamine/xylazine anesthesia, 5 days before administration of bleomycin and delivered for 3 weeks. Mice were weighed daily, sacrificed on day 21 and bronchial fluid was collected. Fibrosis was measured by staining with Sirius red as described for ladostigil. TNFα and IL-6 in the bronchial fluid lavage was measured by ELISA as described for ladostigil.

### Example 1

### Treatment with Ladostigil Reduces LPS-Induced Cytokine Release

Gene expression, synthesis, and secretion of TNFα and IL1β proteins are controlled by independent mechanisms (Shamash et al. 2002). Therefore, to obtain secretion of all three cytokines from microglia the inventor performed a new study using a combination of LPS and 2′-3′-O-(4-benzoyl benzoyl) adenosine 5′-triphosphate (BzATP), an agonist of P2x7 receptors. The inventor then used RNA sequencing (RNA-seq) to perform a detailed analysis of gene expression in the microglia to obtain additional information about the cellular processes through which ladostigil regulates cytokine release.

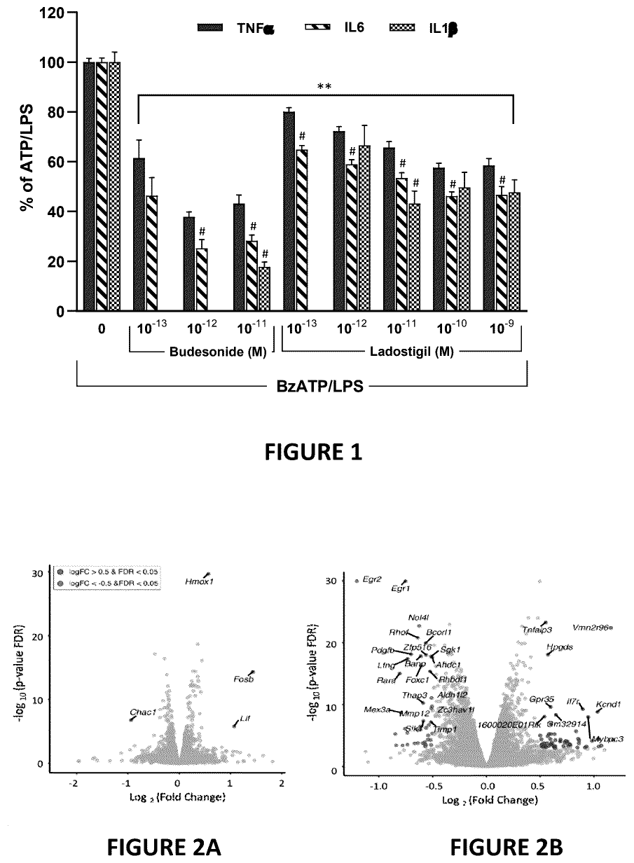

The lowest concentration of ladostigil tested in microglia that significantly decreased cytokine secretion in medium containing BSA and BzATP/LPS was 1×10⁻¹³ M for TNFα and IL6 and 1×10⁻¹² M for IL1β. Maximal reductions of ≈50% for IL6 and IL1β were obtained by ladostigil (1×10⁻¹¹-1×10⁻⁹ M). At all concentrations of ladostigil and budesonide tested, the reductions of IL6 were greater than those of TNF, (p<0.001). Reductions of IL1β by ladostigil (1×10⁻⁹ M) and budesonide (1× 10⁻¹¹ M) was also greater than that of TNFα (FIG. 1).

### Example 2

### Ladostigil Treatment Alters Gene Expression in LPS-Activated Microglia

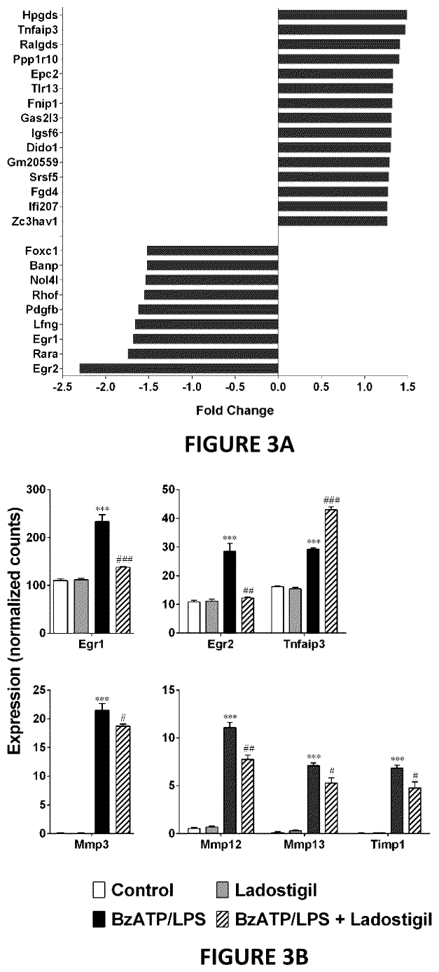

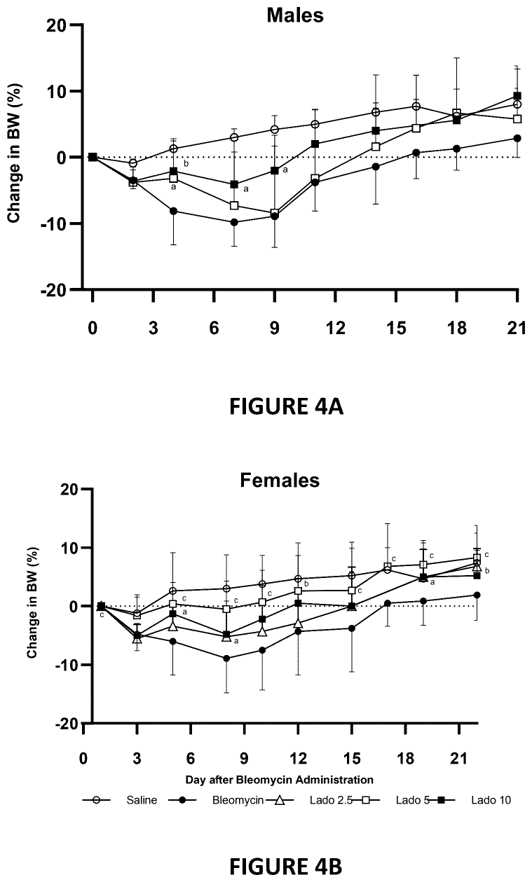

The expression of 25 genes was significantly altered, 8 hrs after their activation by BzATP/LPS (FIGS. 2A-2B), when ladostigil produced its inhibitory effect on cytokine secretion. Among these were: (i) early growth response protein 1 and 2 (Egr1 and Egr2), (ii) matrix metalloproteinase (Mmp) 12 (Mmp12), (iii) tissue inhibitor of metalloprotease 1 (Timp1), all of which increase cytokine release and were downregulated by ladostigil, while TNF alpha-induced protein 3 (TNFαIP3) (A20), which inhibits cytokine release, was up-regulated. (FIGS. 3A-3B).

The addition of BzATP/LPS to microglia increased cytoplasmic levels of TNFαIP3 protein and nuclear ERGR1 protein. Ladostigil (1× 10⁻¹⁰ M) caused a further increase in TNFαIP3 protein (Table 1) but decreased EGR1 (Table 2).

TABLE 1
Effect of ladostigil on levels of TNFAIP3 in microglial cytoplasm

| Treatment | N | Mean conc. ± SEM |
|---|---|---|
| Medium | 32 | 19.8 ± 0.6 |
| BzATP/LPS | 53 | 23.3 ± 1.0* |
| BzATP/LPS + Ladostigil | 58 | 28.8 ± 0.9***^{###} |

TABLE 2
Ladostigil decreases nuclear levels of EGR1
in microglia activated by Bz/ATP and LPS

| | | Mean percent of nuclei with positive |
|---|---|---|
| Treatment | N | EGR1 protein immunoreactivity ± SEM |
| Medium | 32 | 19.0 ± 4.2 |
| BzATP/LPS | 53 | 82.3 ± 3.7*** |
| BzATP/LPS + Ladostigil | 58 | 56.0 ± 3.9***^{###} |

Lado/BzATP/LPS,
***p < 0.001;
BzATP/LPS vs Lado/BzATP/LPS,
^{###}p < 0.001.

### Example 3

### Ladostigil has Anti-Fibrotic Effect on Lung Fibrosis

Thirty-one percent (31%) of males (5/16) males given bleomycin alone died on days 16-19. Twenty-two percent (22%) (2/9) given bleomycin+ladostigil 5 mg/kg and 9% (1/11) given ladostigil 10 mg/kg died on day 17. Among the females, 16% (6/38) given bleomycin alone, died between days 7 and 17. Of those given ladostigil+bleomycin, 2/10 (20%) died after a dose of 2.5 mg/kg, 1/20 (5%) of those given 5 mg/kg and 1/11. The lower mortality of females is consistent with the finding of others and is why many only use female mice for their studies (Sklepkiewicz et al. 2022; Ju et al. 2022).

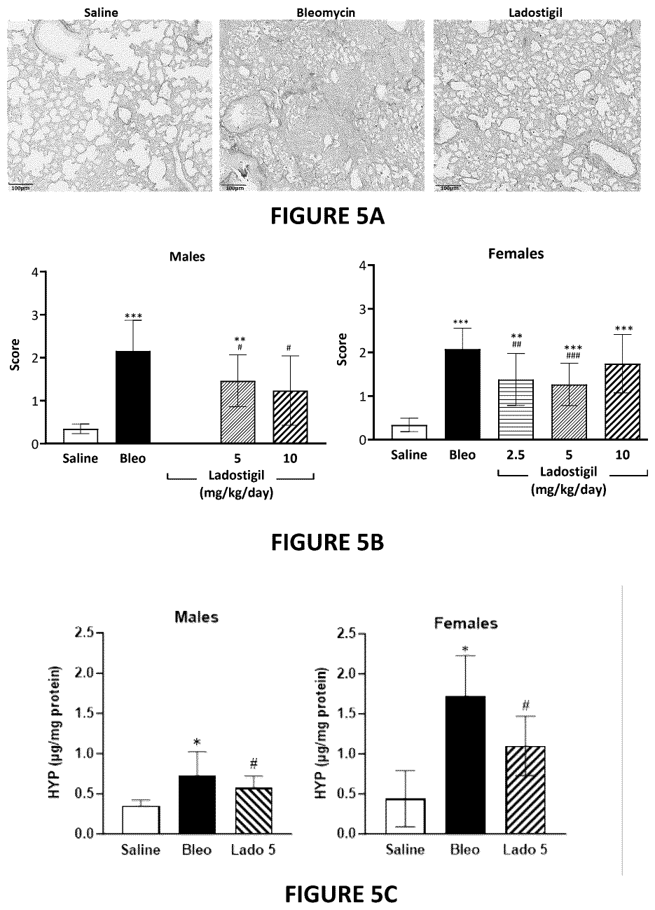

Unless otherwise stated, all results are expressed as mean±STD. Ladostigil significantly reduced fibrosis in males at doses of 5 and 10 mg/kg/day and in females, at doses of 2.5, and 5 mg/Kg/day (FIG. 5B). Ladostigil (5 mg/Kg/day) reduced collagen deposition in mice of both sexes (FIG. 5C).

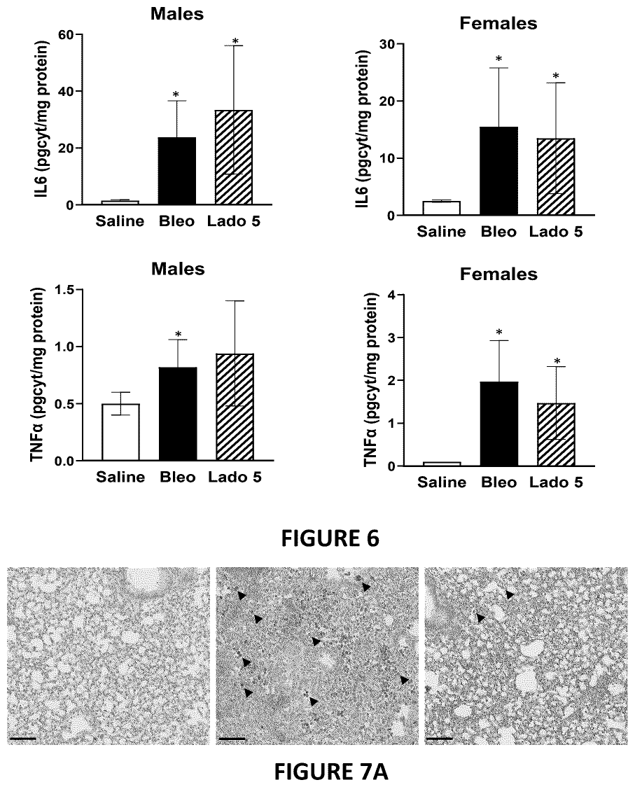

In FIG. 6 the effect of bleomycin and ladostigil on the levels of the pro-inflammatory cytokines, TNFα and IL-6 in the bronchial fluid, is shown. These were generally not affected by ladostigil.

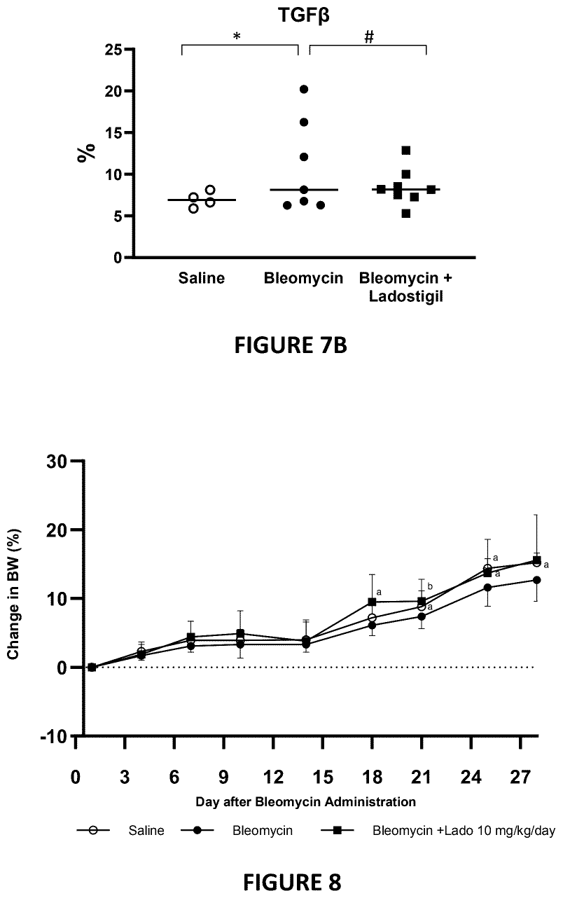

Further, the inventor showed that ladostigil significantly reduced the number of cells containing TGFβ (FIG. 7).

Based on the current data, while ladostigil was shown to reduce fibrosis, it appears it does not have an anti-inflammatory effect in bleomycin-induced lung fibrosis model.

### Example 4

### Ladostigil has a Therapeutic Effect on Skin Sclerosis

The inventor has further examined whether ladostigil has a therapeutic or curative effect on skin sclerosis, as described above (see ‘Materials and methods’).

Weight gain in mice administered with ladostigil was greater than in those on bleomycin on days 18, 21, 24 and 27 (FIG. 8).

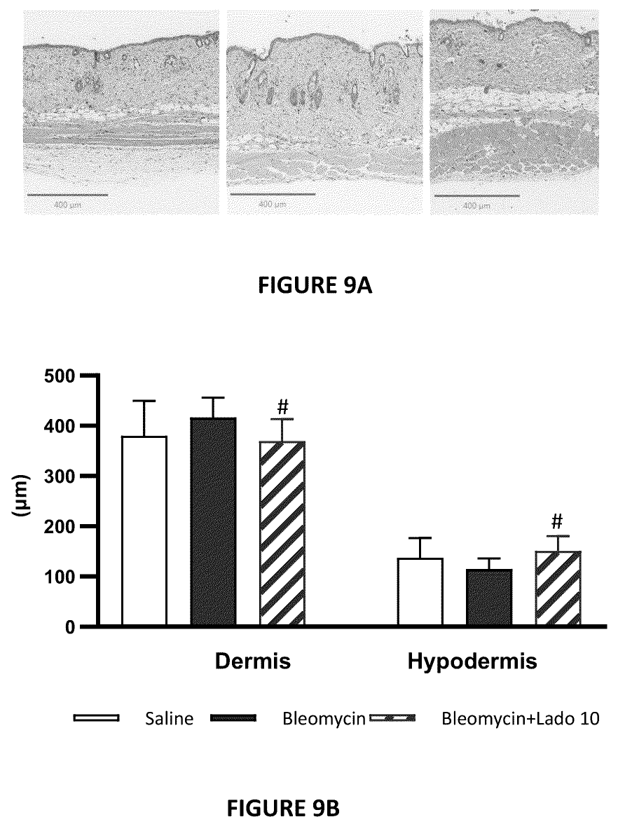

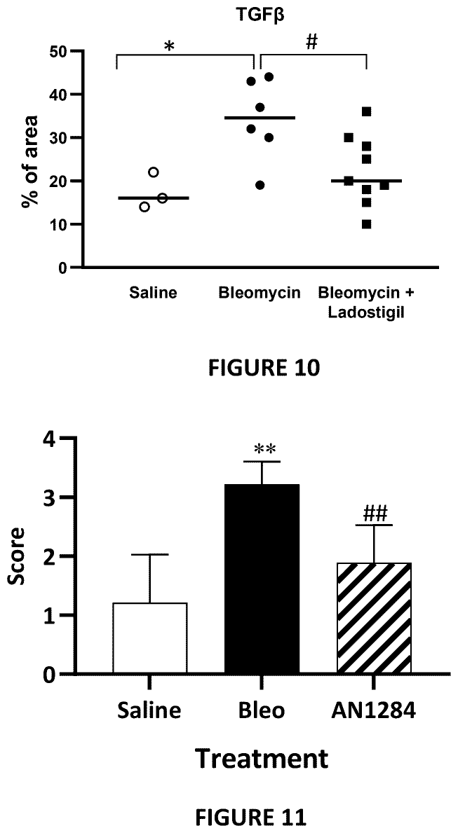

Upon study termination, histological analysis was conducted as described above. To this end, bleomycin treatment induced thickening in conjunction with thinning of the hypodermis, both hallmarks of skin sclerosis. The results show that ladostigil has a therapeutic effect, as evidenced by significant reduction of dermal thickness and increase of hypodermal thickness in mice treated with ladostigil (FIG. 9). Further, the inventor showed that ladostigil treatment significantly reduced the number of TGFβ-containing cells in the skin of ladostigil-treated mice (FIG. 10), corroborating the results achieved in the lungs (FIG. 7).

### Example 5

### AN1284 Decreases Oxidative Stress in Activated Macrophages

Lipopolysaccharide (LPS) is a prototypical endotoxin that binds to the CD14/TLR4/MD2 receptor complex in macrophages, like RAW 264.7 cells. It promotes the secretion of pro-inflammatory cytokines and also increases superoxide, a major reactive oxygen species.

Nrf2 regulates the action of antioxidant proteins that can help protect against oxidative damage. Antioxidants like AN1284 decrease the expression of Nrf2 by lowering oxidative stress.

LPS in the presence of ATP activates NLRP3 and converts procaspase-1 to caspase-1. This enables the processing and secretion of IL1β and other pro-inflammatory cytokines (Katsumoto et al, 2018). LPS also increases early growth response protein EGR1 that increases the release of pro-inflammatory cytokines.

AN1284 decreased the expression of Nrf2 (Table 3). In RAW cells, as in microglia, LPS increased the nuclear translocation of early growth response protein (EGR1) that increases the release of pro-inflammatory cytokines. Like ladostigil in microglial cells, AN1284 reduced ERG1 in macrophages (Table 3). As the case of Nrf2, AN1284 was found to effectively reduce expression of NLRP3 (Table 3).

TABLE 3
AN1284 decreases measures of oxidative stress and
inflammation in RAW 246.7 cells activated by LPS

| Treatment | Nrf2 | NLRP3 | Egr1 |
|---|---|---|---|
| LPS + vehicle | 2.3 | 1.6 | 2.7 |
| LPS + AN1284 | 1.6 | 1.1 | 1.3 |
| 0.1 nM | | | |

### Example 6

### AN1284 for Treating Fibrosis

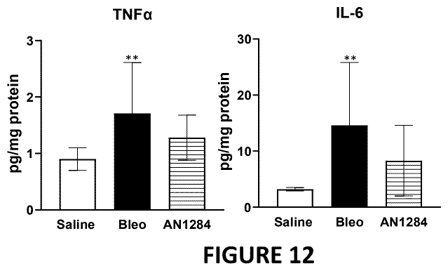

AN1284 reduced body weight loss on days 11 and 14 after bleomycin administration. AN1284 was also found to reduce collagen levels, reflected by reduced fibrosis score (FIG. 11). Further, the results show that AN1284 does not affect levels of pro-inflammatory cytokines in bronchial fluid (FIG. 12).

Based on the current data, while ladostigil and AN1284 were shown to reduce fibrosis, it appears they did not reduce the anti-inflammatory cytokines TNFα, and IL-6 effect in the bronchial fluid of the bleomycin-induced lung fibrosis murine model.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

1. A method of treating or preventing fibrosis in a subject in need thereof, the method comprising administering to said subject a therapeutically effective amount of ladostigil, AN1284, or a combination thereof, thereby treating or preventing fibrosis in the subject.

2. The method of claim 1, wherein said fibrosis is of a tissue selected from: lung, skin, liver, heart, retinal, mediastinal, retroperitoneal cavity, bone marrow, or any combination thereof.

3. The method of claim 1, wherein said fibrosis comprises lung fibrosis, skin sclerosis, or both.

4. The method of claim 1, wherein said administering comprises administering a pharmaceutical composition comprising a therapeutically effective amount of said ladostigil, AN1284, or a combination thereof, and a pharmaceutically acceptable carrier.

5. The method of claim 1, wherein said treating comprises reducing level of transforming growth factor beta (TGFβ) in said subject.

6. The method of claim 1, wherein said treating comprises maintaining levels of TNFα, Interleukin (IL) 6, or both, in said subject.

7.-10. (canceled)

11. The method of claim 5, wherein said reducing level of said TGFβ comprises reducing expression, secretion, amount, abundance, or any combination thereof, of said TGFβ.

12. The method of claim 11, wherein said expression comprises any one of: gene expression level, RNA expression level, and protein expression level.

13. The method of claim 1, further comprising a step comprising determining expression level of TGFβ in said subject.

14. The method of claim 5, wherein said reducing is compared to a control non-treated subject.
